(19) United States
(12) Patent Application Publication
(10) Pub. No.: US 2024/0189262 A1
(43) Pub. Date: Jun. 13, 2024

(54) ANTIBACTERIAL DRESSING, METHOD FOR PREPARING THE SAME, AND APPLICATION THEREOF

(71) Applicant: Suzhou Microport Regenerative Medicine Technology Co. Ltd, Suzhou, Jiangsu Province (CN)

(72) Inventors: Qiang ZHANG, Suzhou (CN); LEI SHI, Suzhou (CN); Dongmei FAN, Suzhou (CN); Jingwan LUO, Suzhou (CN); Chuqi CHENG, Suzhou (CN)

(21) Appl. No.: 18/532,935

(22) Filed: Dec. 7, 2023

(30) Foreign Application Priority Data
Dec. 8, 2022 (CN) 202211572518.3

(51) Int. Cl. A61K 31/155; A61K 9/70; A61K 33/14; A61K 33/30; A61K 47/42; A61P 31/04

(52) U.S. Cl. CPC A61K 31/155; A61K 9/7007; A61K 33/14; A61K 33/30; A61K 47/42; A61P 31/04

(57) ABSTRACT

The present application relates to an antibacterial dressing including an antibacterial composition. The antibacterial composition includes a biguanidine antibacterial agent, a zinc-containing compound, and a magnesium-containing compound with a mass ratio of (0.02 to 1):(0.1 to 0.5):(1 to 5). The antibacterial dressing could be applied onto a skin wound for promoting would healing.

### CROSS-REFERENCE TO RELATED APPLICATIONS

This application claims priority to Chinese patent application No. 202211572518.3, filed on Dec. 8, 2022, the content of which is hereby incorporated by reference in its entirety.

### TECHNICAL FIELD

The present disclosure relates to the field of antibiosis, and particularly to an antibacterial composition, an antibacterial dressing, a method for preparing the same, and application thereof.

### BACKGROUND

The skin, the largest organ in the body, can protect the body from pathogen invasion. When the skin is injured, prompt and proper treatment is essential for the skin to maintain its normal functions. For slight skin injuries, simple disinfection treatment can help the injured skin heal on its own. However, for susceptible and refractory wounds, such as burns, scalds, bedsores, or ulcers, antibacterial dressings are needed to repair the injured skin.

An antibacterial dressing generally consists of two parts, i.e., a dressing and an antibacterial substance. There are many kinds of dressings, and with the progress of technology, more and more new dressings continue to emerge with improved performance. A collagen dressing is a kind of bioactive dressing made of collagen which is extracted from animal tissues. The collagen dressing not only has excellent biodegradability and biocompatibility, but also can accelerate wound healing, therefore which is widely used in the field of skin tissue engineering. The antibacterial substance is crucial to prevent bacterial infection in the antibacterial dressing. Currently, the antibacterial substances that have been used in the collagen dressings include antibiotics, silver antibacterial agents, amino polysaccharides, quaternary ammonium salts, and biguanidine antibacterial agents. The introduction of these substances can have different degrees of antibacterial effects, but there are also some deficiencies. For example, the widespread use of antibiotics has led to bacterial resistance, and silver antibacterial agents lack specificity in targeting bacteria and cells; amino polysaccharides are effective only within specific pH ranges; and quaternary ammonium salts are likely to decompose to produce toxic substances, and bacteria are prone to developing resistance to them. The biguanidine antibacterial agents, especially polyhexamethylene biguanidine hydrochloride (PHMB), are considered to be the safest and most effective antibacterial agents currently available. PHMB is a polycationic hydrochloride, which can form a cationic protective film on the surface of a medium to inhibit the growth of bacteria. However, PHMB has high cytotoxicity while inhibiting bacteria.

### SUMMARY

In view of the above, there is a need to provide an antibacterial composition with low cytotoxicity while having good antibacterial performance.

Moreover, there is a need to provide an antibacterial dressing, a method for preparing the same, and its application.

An antibacterial composition includes a biguanidine antibacterial agent, a zinc-containing compound, and a magnesium-containing compound with a mass ratio of (0.02 to 1):(0.1 to 0.5):(1 to 5).

In an embodiment, the mass ratio of the biguanidine antibacterial agent, the zinc-containing compound, and the magnesium-containing compound is (0.02 to 0.5):(0.2 to 0.5):(2 to 5).

In an embodiment, the antibacterial composition satisfies at least one of the following conditions:
1) the biguanidine antibacterial agent is selected from the group consisting of polyhexamethylene biguanidine hydrochloride, chlorhexidine (i.e., dichlorophenylbiguanidine hexane), dodine (i.e., dodecylguanidine acetate), and any combination thereof;
2) the zinc-containing compound is selected from the group consisting of zinc chloride, zinc sulfate, zinc acetate, zinc nitrate, and any combination thereof;
3) the magnesium-containing compound is selected from the group consisting of magnesium chloride, magnesium sulfate, magnesium phosphate, magnesium nitrate, and any combination thereof.

An antibacterial dressing includes a dressing substrate and an antibacterial layer applied to the dressing substrate, wherein the raw material of the antibacterial layer includes the antibacterial composition as described above.

In an embodiment, the material of the dressing substrate includes collagen.

In an embodiment, the water-absorption ratio of the dressing substrate is less than or equal to 52.71.

A method for preparing an antibacterial dressing, includes the following steps: dissolving the antibacterial composition as described above to obtain a composite solution including free zinc ions, free magnesium ions, and a biguanidine antibacterial agent; and applying the composite solution to a dressing substrate and drying the dressing substrate applied with the composite solution to obtain the antibacterial dressing.

In an embodiment, the pH value of the composite solution is ranged from 4.5 to 6.5.

In an embodiment, the mass of the biguanidine antibacterial agent is 0.02% to 1% of the mass of the composite solution, the mass of the zinc-containing compound is 0.1% to 0.5% of the mass of the composite solution, and the mass of the magnesium-containing compound is 1% to 5% of the mass of the composite solution.

In an embodiment, the step of applying the composite solution to the dressing substrate includes a step of immersing the dressing substrate in the composite solution for 30 minutes (min) to 360 min.

In an embodiment, the mass ratio of the dressing substrate to the composite solution is 1:17 to 1:52.71.

In an embodiment, the type of drying is selected from the group consisting of natural drying, blast drying, freeze drying, vacuum drying, critical point drying, and any combination thereof.

The present application also protects a method for using the antibacterial dressing as described above comprising applying the antibacterial dressing onto a skin wound for promoting wound healing.

During the analysis of skin wounds healing process, the inventors found that the prerequisite for skin healing is to reduce infection, restore skin barrier function, and accelerate the remodeling of the injured skin tissue. During the process of skin healing, fibroblasts are key cells for tissue remodeling, and zinc and magnesium ions play an important regulatory role on the fibroblast function. Magnesium ions can improve cell viability and promote cell proliferation and migration. Zinc ions participate in metabolism and activation of certain enzymes as coenzymes, regulating cell metabolism, antagonizing oxidative stress of cells, and stabilizing intracellular antioxidant enzyme activity, thereby repairing injured skin and mucosae and accelerating the healing of skin tissues. Therefore, in the antibacterial composition as described above, the biguanidine antibacterial agent is modified with zinc- and magnesium-containing compounds, and the ratio of the biguanidine antibacterial agent, the zinc-containing compound, and the magnesium-containing compound is carefully controlled. Therefore, the antibacterial composition with high antibacterial efficacy and low cytotoxicity is obtained.

### BRIEF DESCRIPTION OF THE DRAWINGS

FIG. 1 shows the inhibition effects of the antibacterial dressing prepared in Example 1 against (A) Escherichia coli, (B) Staphylococcus aureus, and (C) Pseudomonas aeruginosa.

FIG. 2 shows the statistical inhibition effects of the antibacterial dressing prepared in Example 1 against (A) Escherichia coli, (B) Staphylococcus aureus, and (C) Pseudomonas aeruginosa.

FIG. 3 shows the cytotoxicity effects of the antibacterial dressings prepared in Example 1 and control group.

FIG. 4A shows the morphology of the fibroblasts cultured on the antibacterial dressing prepared in Example 1.

FIG. 4B shows the morphology of the fibroblasts cultured on the antibacterial dressings prepared in the comparative examples.

### DETAILED DESCRIPTION

The present disclosure will now be described in detail with reference to the specific embodiments in order to facilitate understanding of the present disclosure. The specific embodiments illustrate some examples of the present disclosure. However, the present disclosure may be implemented in many different forms and is not limited to the embodiments described herein. In contrast, these embodiments provided herein are only for understanding the disclosure of the present disclosure more thoroughly and comprehensively.

Unless otherwise indicated, all the technical and scientific terms herein shall be understood as the same meaning as those commonly accepted by those skilled in the art. The terms used herein in the descriptions of the present disclosure are only for the purpose of illustrating specific embodiments, and are not intended to limit the present disclosure.

In the present disclosure, “one or more” refers to any one, two, or more of the listed items, and “several” refers to any two or more of the listed items.

In the present disclosure, an open-end description for the technical features includes not only a close-ended technical solution consisting of the listed features, but also an open-ended technical solution including the listed features.

In the present disclosure, unless otherwise indicated, a numerical interval includes two endpoints of the numerical interval.

In the present disclosure, unless otherwise indicated, a percentage concentration refers to the final concentration. The final concentration is the proportion of a component in a system after adding that component.

When a numerical range is disclosed herein, the range as described is considered continuous and includes the minimum and maximum values of the range, as well as each value between the minimum and maximum values. Further, when the range is for integers, each integer between the minimum and maximum values of the range is included. Furthermore, when a plurality of ranges describing features or characteristics are provided, the ranges can be combined. In other words, unless otherwise indicated, all ranges disclosed herein are to be understood as including any and all sub-ranges included therein.

An “embodiment” in the present description means that specific features, structures, or characteristics described with reference to the embodiment can be included in at least one embodiment of the present disclosure. The terms mentioned in various places in the description do not necessarily refer to the same embodiment, nor are they independent or alternative embodiments mutually exclusive with other embodiments. It can be explicitly and implicitly understood by those skilled in the art that the embodiments described herein can be combined with other embodiments.

In order to address the problem of strong cytotoxicity in the preparation of the collagen antibacterial dressing using PHMB as an antibacterial substance, the present disclosure provides an antibacterial composition, an antibacterial dressing, a method for preparing the same, and its application.

In an embodiment of the present disclosure, an antibacterial composition includes a biguanidine antibacterial agent, a zinc-containing compound, and a magnesium-containing compound with a mass ratio of (0.02 to 1):(0.1 to 0.5):(1 to 5).

In a specific embodiment, the mass ratio of the biguanidine antibacterial agent, the zinc-containing compound, and the magnesium-containing compound can be 0.02:0.1:1, 0.5:0.1:1, 1:0.1:1, 0.02:0.5:1, 0.5:0.5:1, 1:0.5:1, 0.02:0.1:5, 0.5:0.1:5, 1:0.1:5, 0.02:0.5:5, 0.5:0.5:5, 1:0.5:5, or within a range defined by any two values thereabove. Alternatively, the mass ratio of the biguanidine antibacterial agent, the zinc-containing compound, and the magnesium-containing compound can be (0.02 to 1):(0.2 to 0.5):(2 to 5), (0.02 to 0.5):(0.1 to 0.5):(1 to 5), (0.05 to 1):(0.1 to 0.5):(1 to 5), (0.05 to 1):(0.2 to 0.5):(2 to 5), (0.05 to 0.5):(0.1 to 0.5):(1 to 5), or (0.05 to 1):(0.2 to 0.5):(2 to 5).

Further, the mass ratio of the biguanidine antibacterial agent, the zinc-containing compound, and the magnesium-containing compound can be (0.02 to 0.5):(0.2 to 0.5):(2 to 5).

In some embodiments, the biguanidine antibacterial agent is selected from the group consisting of polyhexamethylene biguanidine hydrochloride, dichlorophenylbiguanidine hexane (i.e., chlorhexidine), dodecylguanidine acetate (i.e., dodine), and any combination thereof.

In some embodiments, the zinc-containing compound is selected from the group consisting of zinc chloride, zinc sulfate, zinc acetate, zinc nitrate, and any combination thereof. These substances all have the effect of reducing cytotoxicity. In a specific embodiment, the zinc-containing compound is zinc chloride.

In some embodiments, the magnesium-containing compound is selected from the group consisting of magnesium chloride, magnesium sulfate, magnesium phosphate, magnesium nitrate, and any combination thereof. These substances all have the effect of reducing cytotoxicity. In a specific embodiment, the magnesium-containing compound is magnesium chloride.

In some embodiments, the antibacterial composition includes polyhexamethylene biguanidine hydrochloride, zinc chloride, and magnesium chloride with a mass ratio of (0.02 to 1):(0.1 to 0.5):(1 to 5). Further, the antibacterial composition includes polyhexamethylene biguanidine hydrochloride, zinc chloride, and magnesium chloride with a mass ratio of (0.02 to 0.5):(0.2 to 0.5):(2 to 5).

During the analysis of skin wound healing process, the inventors found that the prerequisite for skin healing is to reduce infection, restore skin barrier function, and accelerate the remodeling of the injured skin tissue. During the process of skin healing, fibroblasts are key cells for tissue remodeling, and zinc and magnesium ions play an important regulatory role on the fibroblast function. Magnesium ions can improve cell viability and promote cell proliferation and migration. Zinc ions participate in metabolism and activation of certain enzymes as coenzymes, regulating cell metabolism, antagonizing oxidative stress of cells, and stabilizing intracellular antioxidant enzyme activity, thereby repairing injured skin and mucosae and accelerating the healing of skin tissues. Therefore, the biguanidine antibacterial agent is modified with the zinc-containing compound and the magnesium-containing compound, and the ratio of the biguanidine antibacterial agent, the zinc-containing compound, and the magnesium-containing compound is carefully controlled. Therefore, the antibacterial composition with high antibacterial efficacy and low cytotoxicity is obtained, which can weaken the cytotoxicity of the biguanidine antibacterial agent while retaining the antibacterial ability of the biguanidine antibacterial agent.

By adjusting the ratio of the components within the antibacterial composition, the zinc-containing compound and the magnesium-containing compound could be combined in sufficient quantities with the biguanidine antibacterial agent. Under the condition that the antibacterial composition is applied in preparing an antibacterial dressing, the obtained antibacterial dressing would be soft and fit, residue-free, without wrinkles or cracking.

An embodiment of the present disclosure further provides an antibacterial dressing, including a dressing substrate and an antibacterial layer applied to the dressing substrate. The raw material of the antibacterial layer includes the antibacterial composition as described above.

In some embodiments, the material of the dressing substrate includes collagen. The collagen dressing is a kind of bioactive dressings made from collagen that is extracted from animal tissues. The collagen dressing not only has excellent biodegradability and biocompatibility, but also can accelerate the wound healing.

In some embodiments, the collagen can be derived from bovine Achilles tendon, and the average molecular weight of the collagen is ranged from 50,000 to 300,000.

In an embodiment, the dressing substrate is prepared as follows. Step one, pretreating and slicing a bovine Achilles tendon; step two, the sliced bovine Achilles tendon being crushed and homogenized, and then added into an acetic acid solution containing pepsin for low-temperature stirring and extracting; step three, centrifuging to collect the supernatant; step four, salting out the supernatant and filtering to obtain crude collagen extract; step five, re-dissolving the crude collagen extract with an acetic acid solution and purifying it to obtain a concentrated collagen solution; and step six, freeze-drying the concentrated collagen solution to remove moisture, and obtaining the dressing substrate.

In some embodiments, the water-absorption ratio of the dressing substrate is less than or equal to 52.71. In a specific embodiment, the test method for water-absorption ratio refers to YY/T 1511-2017 collagen sponge, as follows:

Taking a sample with a mass of approximately 20 mg, denoted as m1. Immersing the sample in a beaker filled with water having a temperature of 20±1° C., and then gently kneading with fingers until the sample is completely immersed, and all air is removed. After absorbing enough water, gently clamping one corner of the sample with small tweezers to lift it from the water. Gently holding the tweezers and draining it for 1 min. Then weighing again, denoted as m2. The water-absorption ratio is calculated according to the following equation.

A=(m2−m1)/m1

In the equation, A is the water-absorption ratio of the sample; m1 is the mass (g) of the sample before immersing, and m2 is the mass (g) of the sample after immersion.

In some embodiments, the antibacterial layer is applied on the surface of the dressing substrate, applied inside the dressing substrate, or applied both on the surface of the dressing substrate and inside the dressing substrate.

The antibacterial dressing as described above has at least the following advantages.

First, in the antibacterial dressing as described above, the biguanidine antibacterial agent is modified with the zinc-containing compound and the magnesium-containing compound, and the ratio of the biguanidine antibacterial agent, the zinc-containing compound, and the magnesium-containing compound is carefully controlled. As such, the antibacterial composition with high antibacterial efficacy and low cytotoxicity is obtained. In this way, the cytotoxicity of the biguanidine antibacterial agent can be weakened or addressed, while the antibacterial ability of the biguanidine antibacterial agent can be retained.

Second, the antibacterial composition is combined with collagen to prepare a new collagen antibacterial dressing with strong antibacterial ability and good cytocompatibility.

An embodiment of the present disclosure further provides a method for preparing the antibacterial dressing, including the following steps.

Step S110, dissolving the antibacterial composition to obtain a composite solution including free zinc ions, free magnesium ions, and the biguanidine antibacterial agent.

The antibacterial composition has been described above, and will not be repeated.

In some embodiments, the pH of the composite solution is ranged from 4.5 to 6.5. Ensure that zinc and magnesium ions in their free state can bind to the biguanidine antibacterial agents at the above pH, and avoid causing tissue inflammation or collagen dissolution if the pH is too low, or causing precipitation of zinc and magnesium ions or partial denaturation of collagen and loss of biological activity if the pH is too high.

In some embodiments, the mass of the biguanidine antibacterial agent can be 0.02% to 1% of the mass of the composite solution. For example, the mass of the biguanidine antibacterial agent can be, but is not limited to, 0.02%, 0.05%, 0.1%, 0.2%, 0.3%, 0.4%, 0.5%, 0.6%, 0.7%, 0.8%, 0.9%, 1%, or within any range defined by these values, of the mass of the composite solution. Optionally, the mass of the biguanidine antibacterial agent can be 0.02% to 0.5%, 0.05% to 1%, or 0.05% to 0.5%, etc. of the mass of the composite solution.

In some embodiments, the mass of the zinc-containing compound can be 0.1% to 0.5% of the mass of the composite solution. For example, the mass of the zinc-containing compound can be, but is not limited to, 0.1%, 0.15%, 0.2%, 0.25%, 0.3%, 0.35%, 0.4%, 0.45%, 0.5%, or within any range defined by these values, of the mass of the composite solution. Optionally, the mass of the zinc-containing compound can be 0.2% to 0.5% of the mass of the composite solution.

In some embodiments, the mass of the magnesium-containing compound can be 1% to 5% of the mass of the composite solution. For example, the mass of the magnesium-containing compound can be, but is not limited to, 1%, 1.5%, 2%, 2.5%, 3%, 3.5%, 4%, 4.5%, 5%, or within any range defined by these values, of the mass of the composite solution. Optionally, the mass of the magnesium-containing compound can be 2% to 5% of the mass of the composite solution.

In some embodiments, the solvent in the composite solution can be water.

Step S120, applying the composite solution to a dressing substrate and drying to obtain the antibacterial dressing.

In some embodiments, in applying the composite solution to the dressing substrate, the dressing substrate is immersed in the composite solution.

In an embodiment, the dressing substrate is immersed in the composite solution for 30 min to 360 min.

In some embodiments, the water-absorption ratio of the dressing substrate is less than or equal to 52.71. Ensure that a sufficient amount of the composite solution can be applied to the collagen dressing under such water-absorption ratio.

In some embodiments, the mass ratio of the dressing substrate to the composite solution is 1:17 to 1:52.71. Under the above mass ratio, the collagen dressing can sufficiently absorb the composite solution without causing the collagen dressing to stack and wrinkle if the system is too large, thereby affecting the shape and performance of the product.

In some embodiments, the drying type can be selected from the group consisting of natural drying, blast drying, freeze drying, vacuum drying, critical point drying, and any combination thereof.

In some the embodiments, the method further includes a step of preparing the dressing substrate. In a specific embodiment, the material of the dressing substrate can be collagen. In some embodiments, the collagen is bovine Achilles tendon collagen, with an average molecular weight of 50,000 to 300,000.

In an embodiment, the step of preparing the dressing substrate includes extracting collagen from bovine Achilles tendon as raw material, and drying to obtain the collagen dressing substrate.

Further, the dressing substrate is prepared as follows. Step one, pretreating and slicing a bovine Achilles tendon; step two, the sliced bovine Achilles tendon being crushed and homogenized, and then added into an acetic acid solution containing pepsin for low-temperature stirring and extracting; step three, centrifuging to collect the supernatant; step four, salting out the supernatant and filtering to obtain crude collagen extract; step five, re-dissolving the crude collagen extract with an acetic acid solution and purifying it to obtain a concentrated collagen solution; and step six, freeze-drying the concentrated collagen solution to remove moisture, thereby obtaining the dressing substrate.

The method for preparing the antibacterial dressing as described above has at least the following advantages.

First, in the method for preparing the antibacterial dressing as described above, the biguanidine antibacterial agent is modified with the zinc-containing compound and the magnesium-containing compound, and the ratio of the biguanidine antibacterial agent, the zinc-containing compound, and the magnesium-containing compound is carefully controlled. As such, the antibacterial composition with high antibacterial efficacy and low cytotoxicity is obtained. The method can weaken or address the cytotoxicity of the biguanidine antibacterial agent while retaining the antibacterial ability of the biguanidine antibacterial agent.

Second, in the method for preparing the antibacterial dressing as described above, the antibacterial composition is combined with collagen to prepare a new collagen antibacterial dressing with strong antibacterial ability and good cytocompatibility.

An embodiment of the present disclosure further provides a method for using the antibacterial dressing as described above comprising using the antibacterial dressing.

In order to make the purposes and advantages of the present disclosure more clear, the antibacterial dressing and the effects thereof of the present disclosure will be detailed in combination with the specific examples. It shall be understood that the specific examples described herein are intended only to illustrate the present disclosure rather than to limit the present disclosure. Unless otherwise indicated, unmentioned components other than unavoidable impurities are not included in the examples. Unless otherwise indicated, the reagents and instruments used in the examples are conventional choices in the field. Experimental methods that do not specified in the examples shall be implemented in accordance with conventional conditions, such as those described in literature, books or methods recommended by the manufacturer.

The materials used in the examples and comparative examples are as follows.

The collagen dressing from bovine Achilles tendon, with the average molecular weight ranged from 50,000 to 300,000, is prepared as follows.

Step one, removing tendon sheath and fascia of the bovine Achilles tendon, and then slicing the bovine Achilles tendon. Step two, preparing an acetic acid solution with a mass percentage concentration of 0.01% pepsin, and a concentration of 0.5 mol/L acetic acid. Step three, crushing the sliced bovine Achilles tendon in step one and homogenizing it. Adding it into the acetic acid solution containing pepsin prepared in step two, stirring at a rotating speed of 200 rpm at 6.8° C. for 24 hours. Step four, centrifuging at 4000 rpm for 20 min to collect the supernatant. Step five, Adding 0.9 mol of solution chloride to the supernatant for salting out, and then filtering to obtain crude collagen extract. Step six, re-dissolving the crude collagen extract with 0.5 mol/L acetic acid solution. Step seven, purifying the re-dissolved crude collagen extract prepared in step six by ultra-filtration for 8 hours to obtain a concentrated collagen solution. Step eight, pouring the concentrated collagen solution into a stainless steel disc and freeze-drying it at −56° C., 28 Pa for 8 hours to remove moisture, thereby obtaining the collagen dressing. The water-absorption ratio of the collagen dressing is 37.15.

PHMB is available from Jinhong (Tianjin) Science and Technology Ltd.

Zinc chloride, magnesium chloride, pepsin, acetic acid, sodium chloride, sodium hydroxide, and hydrochloric acid are available from Sinopharm Chemical Reagent Co., Ltd.

### Example 1

The present example provides a collagen antibacterial dressing. The preparation process of the collagen antibacterial dressing is as follows.
Step one, a collagen dressing is prepared by the method as described above.
Step two, PHMB, zinc chloride, and magnesium chloride are dissolved in deionized water to obtain a composite solution, whose pH value is adjusted to be 5.5. In the composite solution, a mass percentage concentration of PHMB is 0.02%, the mass percentage concentration of zinc chloride is 0.1%, and the mass percentage concentration of magnesium chloride is 1%.
Step three, the collagen dressing prepared in step one is immersed in the composite solution prepared in step two for 120 min to obtain a collagen antibacterial dressing precursor. A mass ratio of the collagen dressing to the composite solution is 1:50.
Step four, the collagen antibacterial dressing precursor prepared in step three is freeze-dried at −56° C., 28 Pa for 8 hours to obtain the collagen antibacterial dressing of the present example.

### Example 2

The present example provides a collagen antibacterial dressing. The specific preparation process of the collagen antibacterial dressing is similar to that in Example 1, except that in the composite solution prepared in step two, the mass percentage concentration of PHMB is 0.5%, the mass percentage concentration of zinc chloride is 0.1%, and the mass percentage concentration of magnesium chloride is 1%. The other steps are the same as those in Example 1.

### Example 3

The present example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 1%, 0.1% and 1% respectively. The other steps are the same as those in Example 1.

### Example 4

The present example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 0.02%, 0.5% and 1% respectively. The other steps are the same as those in Example 1.

### Example 5

The present example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 0.5%, 0.5% and 1% respectively. The other steps are the same as those in Example 1.

### Example 6

The present example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 1%, 0.5% and 1% respectively. The other steps are the same as those in Example 1.

### Example 7

The present example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 0.02%, 0.1% and 5% respectively. The other steps are the same as those in Example 1.

### Example 8

The present example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 0.5%, 0.1% and 5% respectively. The other steps are the same as those in Example 1.

### Example 9

The present example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 1%, 0.1% and 5% respectively. The other steps are the same as those in Example 1.

### Example 10

The present example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 0.02%, 0.5% and 5% respectively. The other steps are the same as those in Example 1.

### Example 11

The present example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 0.5%, 0.5% and 5% respectively. The other steps are the same as those in Example 1.

### Example 12

The present example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 1%, 0.5% and 5% respectively. The other steps are the same as those in Example 1.

### Example 13

The present example provides a collagen antibacterial dressing. The specific preparation process of the collagen antibacterial dressing is similar to that in Example 1, except that in the composite solution prepared in step two, zinc chloride is replaced with zinc sulfate, and magnesium chloride is replaced with magnesium sulfate.

### Example 14

The present example provides a collagen antibacterial dressing. The specific preparation process of the collagen antibacterial dressing is similar to that in Example 1, except that in the composite solution prepared in step two, zinc chloride is replaced with zinc nitrate, and magnesium chloride is replaced with magnesium phosphate.

### Example 15

The present example provides a collagen antibacterial dressing. The specific preparation process of the collagen antibacterial dressing is similar to that in Example 1, except that in the composite solution prepared in step two, zinc chloride is replaced with zinc acetate, and magnesium chloride is replaced with magnesium nitrate.

### Comparative Example 1

The present comparative example provides a collagen antibacterial dressing. The specific preparation process of the collagen antibacterial dressing is as follows.
Step one, a collagen dressing is prepared by the method as described above.
Step two, PHMB is dissolved in deionized water to obtain a PHMB solution, whose pH value is adjusted to be 5.5. The mass percentage concentration of PHMB is 0.02%.
Step three, the collagen dressing prepared in step one is immersed in the PHMB solution prepared in step two for 120 min to obtain a collagen antibacterial dressing precursor. The mass ratio of the collagen dressing to the PHMB solution is 1:55.
Step four, the collagen antibacterial dressing precursor prepared in step three is freeze-dried at −56° C., 28 Pa for 8 hours to obtain the collagen antibacterial dressing of the present comparative example.

### Comparative Example 2

The present Comparative Example provides a collagen antibacterial dressing. The specific preparation process of the collagen antibacterial dressing is similar to that in Comparative Example 1, except that instead of the PHMB solution, a composite solution including PHMB and zinc chloride is prepared in step two, and the mass percentage concentrations of PHMB and zinc chloride are 0.02% and 0.1%, respectively. The other steps are the same as those in Comparative Example 1.

### Comparative Example 3

The present comparative example provides a collagen antibacterial dressing. The specific preparation process of the collagen antibacterial dressing is similar to that in Comparative Example 1, except that instead of the PHMB solution, a composite solution including PHMB and magnesium chloride is prepared in step two, and the mass percentage concentrations of PHMB and magnesium chloride are 0.02% and 0.1%, respectively. The other steps are the same as those in Comparative Example 1.

### Comparative Example 4

The present comparative example provides a collagen antibacterial dressing. The specific preparation process of the collagen antibacterial dressing is similar to that in Comparative Example 1, except that instead of the PHMB solution, a composite solution including zinc chloride and magnesium chloride is prepared in step two, and the mass percentage concentrations of zinc chloride and magnesium chloride are 0.1% and 1%, respectively. The other steps are the same as those in Comparative Example 1.

### Comparative Example 5

The present comparative example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 1.5%, 0.1%, and 1%, respectively. The other steps are the same as those in Example 1.

### Comparative Example 6

The present comparative example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 0.01%, 0.1% and 1% respectively. The other steps are the same as those in Example 1.

### Comparative Example 7

The present comparative example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 0.02%, 0.05% and 1% respectively. The other steps are the same as those in Example 1.

### Comparative Example 8

The present comparative example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 0.02%, 0.8% and 1% respectively. The other steps are the same as those in Example 1.

### Comparative Example 9

The present comparative example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 0.02%, 0.1% and 0.5% respectively. The other steps are the same as those in Example 1.

### Comparative Example 10

The present comparative example provides a collagen antibacterial dressing. The specific process for preparation of the collagen antibacterial dressing is similar to that in Example 1, except that in the composite solution prepared in step two, the mass percentage concentrations of PHMB, zinc chloride, and magnesium chloride are 0.02%, 0.1% and 6% respectively. The other steps are the same as those in Example 1.

### Comparative Example 11

The present comparative example provides a collagen antibacterial dressing. The specific preparation process of the collagen antibacterial dressing is similar to that in Example 1, except that in the composite solution prepared in step two, the mass percentage concentrations of PHMB, zinc chloride, and magnesium chloride are 0.02%, 1% and 0.1% respectively. The other steps are the same as those in Example 1.

The specific tests are as follows.

### 1. Antibacterial Activity

The collagen antibacterial dressing samples prepared in Examples 1 to 15 and Comparative Examples 1 to 11 are respectively attached to a culture medium inoculated with Escherichia coli (EC), Staphylococcus aureus (SA), or Pseudomonas aeruginosa (PA), and then incubated in a constant temperature incubator at 37° C. for 24 hours. After the incubation, the growth of Escherichia coli (EC), Staphylococcus aureus (SA), and Pseudomonas aeruginosa (PA) are observed, and antibacterial zones (i.e., bacteriostatic circles) are formed surrounding the antibacterial dressings against the three bacteria. The widths of the bacteriostatic circles are measured, which are the distances from the outer edges of the antibacterial dressing samples to the outer edges of the corresponding bacteriostatic circles. When the antibacterial dressing samples are round shaped, the bacteriostatic circles are round circles, and the widths of the bacteriostatic circles are obtained by subtracting the inner radius from the outer radius of the bacteriostatic circles. The results are shown in Table 1 below.

It can be seen from Table 1 that the antibacterial dressing in Comparative Example 1, containing only PHMB, has smaller widths of the bacteriostatic circles as compared to the antibacterial dressing in Comparative Example 4, containing only zinc chloride and magnesium chloride, against EC and SA. The widths of the bacteriostatic circles formed by both of the two antibacterial dressings against PA are zero (0). Within a certain range, increasing the PHMB content in the antibacterial dressings led to a gradual increase in the widths of the bacteriostatic circles against the three types of bacteria. As the content of zinc chloride and/or magnesium chloride increases, the widths of the bacteriostatic circles formed by the antibacterial dressings against the three bacteria are not significantly varied but the antibacterial dressings are wrinkled and cracked, indicating that the antibacterial performance of the antibacterial dressing could be effectively regulated by changing the content of PHMB.

### 2. Cell Viability

The collagen antibacterial dressing samples prepared in Examples 1 to 15 and Comparative Examples 1 to 11 are respectively placed in 24-well culture plates, inoculated with a single cell suspension of fibroblasts, and then cultured in an incubator at 37° C., 5% carbon dioxide for 24 hours. Then, 20 μl CCK8 reagent is added, and the incubation is continued at 37° C. for 4 hours. The culture supernatant is aspirated, and the absorbance value at 450 nm for each supernatant is recorded by using an enzyme-linked immunosorbent assay reader. The results are shown in Table 1. In the present disclosure, the absorbance value of the supernatant at 450 nm is above 0.857, optionally between 0.86 and 1.37.

It can be seen from Table 1 that the antibacterial dressings including PHMB exhibit a certain degree of cytotoxicity. Furthermore, increasing the concentration of PHMB results in a continuous decrease in cell viability. In contrast, the dressings including an appropriate amount of zinc chloride and/or magnesium chloride exhibit higher cell viability values compared to the pure PHMB dressings, indicating that zinc chloride and magnesium chloride can reduce the cytotoxicity of PHMB.

TABLE 1
Composition, antibacterial efficacy, and cell viability of antibacterial
dressings prepared in Examples and Comparative Examples

| | | | | Antibacterial ability | Cell viability | |
|---|---|---|---|---|---|---|
| | | Zinc | Magnesium | index (width of | (absorbance | Morphology |
| | PHMB | chloride | chloride | bacteriostatic circle, | value at 450 | of the |
| | (0.01%) | (0.01%) | (0.01%) | cm) | nm) | dressing |
| Example 1 | 2 | 10 | 100 | EC: 0.38 ± 0.20 | 1.27 ± 0.02 | Loose and |
| | | | | SA: 0.48 ± 0.11 | | porous |
| | | | | PA: 0.09 ± 0.02 | | |
| Example 2 | 50 | 10 | 100 | EC: 0.42 ± 0.10 | 1.16 ± 0.03 | Loose and |
| | | | | SA: 0.55 ± 0.06 | | porous |
| | | | | PA: 0.13 ± 0.02 | | |
| Example 3 | 100 | 10 | 100 | EC: 0.54 ± 0.10 | 1.06 ± 0.06 | Loose and |
| | | | | SA: 0.62 ± 0.09 | | porous |
| | | | | PA: 0.17 ± 0.02 | | |
| Example 4 | 2 | 50 | 100 | EC: 0.42 ± 0.10 | 1.26 ± 0.02 | Loose and |
| | | | | SA: 0.53 ± 0.07 | | porous |
| | | | | PA: 0.17 ± 0.03 | | |
| Example 5 | 50 | 50 | 100 | EC: 0.50 ± 0.15 | 1.05 ± 0.07 | Loose and |
| | | | | SA: 0.59 ± 0.07 | | porous |
| | | | | PA: 0.37 ± 0.03 | | |
| Example 6 | 100 | 50 | 100 | EC: 0.56 ± 0.11 | 1.04 ± 0.04 | Loose and |
| | | | | SA: 0.62 ± 0.07 | | porous |
| | | | | PA: 0.44 ± 0.03 | | |
| Example 7 | 2 | 10 | 500 | EC: 0.44 ± 0.10 | 1.24 ± 0.03 | Dry and |
| | | | | SA: 0.49 ± 0.07 | | cracked |
| | | | | PA: 0.18 ± 0.02 | | |
| Example 8 | 50 | 10 | 500 | EC: 0.48 ± 0.10 | 1.11 ± 0.01 | Dry and |
| | | | | SA: 0.59 ± 0.06 | | cracked |
| | | | | PA: 0.33 ± 0.02 | | |
| Example 9 | 100 | 10 | 500 | EC: 0.54 ± 0.12 | 1.01 ± 0.01 | Dry and |
| | | | | SA: 0.63 ± 0.09 | | cracked |
| | | | | PA: 0.41 ± 0.02 | | |
| Example 10 | 2 | 50 | 500 | EC: 0.43 ± 0.10 | 1.32 ± 0.05 | Dry and |
| | | | | SA: 0.49 ± 0.07 | | cracked |
| | | | | PA: 0.15 ± 0.02 | | |
| Example 11 | 50 | 50 | 500 | EC: 0.53 ± 0.10 | 1.14 ± 0.02 | Dry and |
| | | | | SA: 0.63 ± 0.02 | | cracked |
| | | | | PA: 0.46 ± 0.06 | | |
| Example 12 | 100 | 50 | 500 | EC: 0.62 ± 0.11 | 0.88 ± 0.02 | Dry and |
| | | | | SA: 0.68 ± 0.02 | | cracked |
| | | | | PA: 0.46 ± 0.04 | | |
| Example 13 | 2 | 10 | 100 | EC: 0.41 ± 0.22 | 1.20 ± 0.12 | Loose and |
| | | | | SA: 0.45 ± 0.13 | | porous |
| | | | | PA: 0.11 ± 0.02 | | |
| Example 14 | 2 | 10 | 100 | EC: 0.38 ± 0.27 | 1.32 ± 0.10 | Loose and |
| | | | | SA: 0.49 ± 0.13 | | porous |
| | | | | PA: 0.15 ± 0.06 | | |
| Example 15 | 2 | 10 | 100 | EC: 0.44 ± 0.18 | 1.24 ± 0.11 | Loose and |
| | | | | SA: 0.45 ± 0.11 | | porous |
| | | | | PA: 0.09 ± 0.05 | | |
| Comparative | 2 | 0 | 0 | EC: 0.08 ± 0.02 | 0.36 ± 0.03 | Loose and |
| Example 1 | | | | SA: 0.25 ± 0.01 | | porous |
| | | | | PA: 0 | | |
| Comparative | 2 | 50 | 0 | EC: 0.62 ± 0.07 | 1.18 ± 0.07 | Loose and |
| Example 2 | | | | SA: 0.52 ± 0.07 | | porous |
| | | | | PA: 0 | | |
| Comparative | 2 | 0 | 100 | EC: 0.05 ± 0.003 | 1.15 ± 0.05 | Loose and |
| Example 3 | | | | SA: 0.20 ± 0.01 | | porous |
| | | | | PA: 0 | | |
| Comparative | 0 | 50 | 100 | EC: 0.47 ± 0.12 | 1.33 ± 0.04 | Loose and |
| Example 4 | | | | SA: 0.73 ± 0.05 | | porous |
| | | | | PA: 0 | | |
| Comparative | 150 | 10 | 100 | EC: 0 | 0.03 ± 0.01 | Loose and |
| Example 5 | | | | SA: 0 | | porous |
| | | | | PA: 0 | | |
| Comparative | 1 | 10 | 100 | EC: 0.18 ± 0.08 | 1.24 ± 0.04 | Loose and |
| Example 6 | | | | SA: 0.26 ± 0.14 | | porous |
| | | | | PA: 0.07 ± 0.03 | | |
| Comparative | 2 | 5 | 100 | EC: 0.34 ± 0.12 | 1.10 ± 0.12 | Loose and |
| Example 7 | | | | SA: 0.41 ± 0.15 | | porous |
| | | | | PA: 0.10 ± 0.02 | | |
| Comparative | 2 | 80 | 100 | EC: 0.45 ± 0.11 | 1.28 ± 0.13 | Wrinkled |
| Example 8 | | | | SA: 0.57 ± 0.09 | | |
| | | | | PA: 0.17 ± 0.08 | | |
| Comparative | 2 | 10 | 50 | EC: 0.31 ± 0.13 | 1.27 ± 0.02 | Loose and |
| Example 9 | | | | SA: 0.42 ± 0.12 | | porous |
| | | | | PA: 0.09 ± 0.05 | | |
| Comparative | 2 | 10 | 600 | EC: 0.51 ± 0.14 | 1.33 ± 0.11 | Dry and |
| Example 10 | | | | SA: 0.58 ± 0.07 | | |
| | | | | PA: 0.23 ± 0.08 | | |
| Comparative | 2 | 100 | 10 | EC: 0.39 ± 0.20 | 1.26 ± 0.09 | Wrinkled |
| Example 11 | | | | SA: 0.50 ± 0.11 | | |
| | | | | PA: 0.14 ± 0.06 | | |

When the antibacterial ability index is zero (0), it is indicated that there is almost no antibacterial effect. The larger the width of the bacteriostatic circles, the better the antibacterial effect. The widths of the bacteriostatic circles formed by the antibacterial dressings prepared in Comparative Examples 1 to 4 against PA are all zero (0), indicating that there is no antibacterial effect of the antibacterial dressings prepared in Comparative Examples 1 to 4 against PA.

The assessment of cell viability is determined according to the cell viability value of the pure collagen dressing (i.e., the blank control group, including none of PHMB, zinc chloride, and magnesium chloride). The cell viability value of the blank control group obtained from the test is 0.857, and thus the cell viability is considered poor when the cell viability value is lower than 0.857, and relatively good when the cell viability value is greater than 0.857. In Comparative Examples 1 to 4, only the cell viability value in Comparative Example 1 is lower than 0.857 for the reason that the antibacterial dressing prepared in Comparative Example 1 consists of the collagen dressing and PHMB, and PHMB is cytotoxic.

The antibacterial performance and cell viability of the collagen antibacterial dressings prepared in Comparative Examples 5 to 7 and 9 are inferior to those of the collagen antibacterial dressings prepared in the examples. Although the antibacterial performance and cell viability of the collagen antibacterial dressings prepared in Comparative Examples 8, 10 and 11 are not significantly different from those of the collagen antibacterial dressings prepared in the examples, the collagen antibacterial dressings prepared in Comparative Examples 8, 10 and 11 are dry and cracked or wrinkled, which is not conducive to the practical application of the collagen dressings.

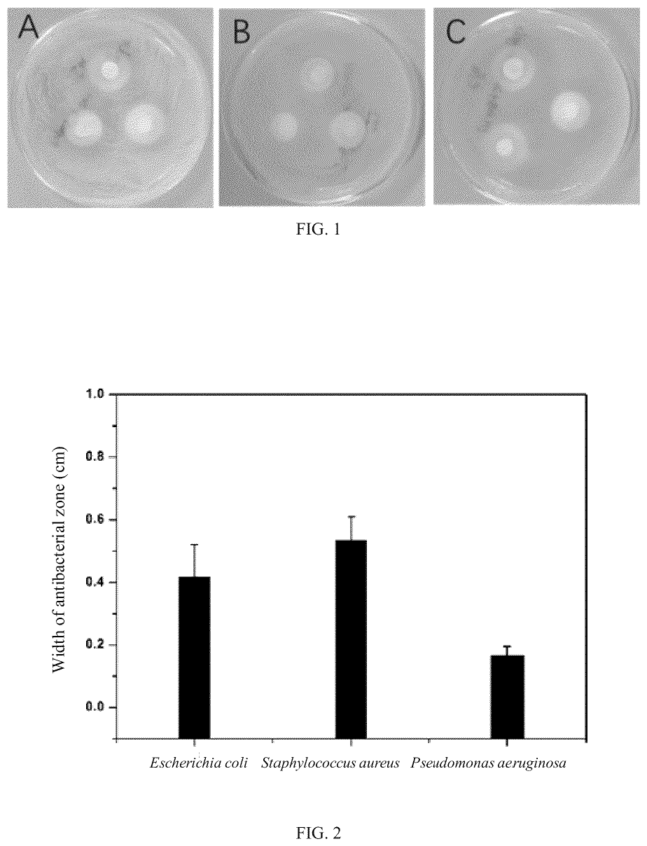

FIG. 1 shows the inhibition results of the antibacterial dressing prepared in Example 1 against (A) Escherichia coli, (B) Staphylococcus aureus, and (C) Pseudomonas aeruginosa. FIG. 1 shows that when placing the antibacterial dressing into a culture medium inoculated with Escherichia coli, Staphylococcus aureus, or Pseudomonas aeruginosa, bacteriostatic circles would be formed surrounding the antibacterial dressing after incubation, which is semi-transparent and formed due to the antibacterial dressings against Escherichia coli, Staphylococcus aureus, or Pseudomonas aeruginosa, each is shown in FIG. 1A, 1B or 1C. In FIG. 1, the inner white, sheet-like materials are the antibacterial dressings including collagen, semi-transparent layers surrounding the sheet-like materials are antibacterial zones which are also called the bacteriostatic circles, and outside the semi-transparent layers is the bacterial growth zone. Within the bacteriostatic circle, bacteria are inhibited to grow. The direct observed result is that the wider the semi-transparent layer, the larger the bacteriostatic circle width. As can be seen from FIG. 1, the collagen antibacterial dressing prepared in Example 1 exhibits relatively wide bacteriostatic circle against (A) Escherichia coli, (B) Staphylococcus aureus, or (C) Pseudomonas aeruginosa. In the present disclosure, after incubated in a constant temperature incubator at 37° C. for 24 hours the width of the bacteriostatic circle formed due to the antibacterial dressing against Escherichia coli is between 0.11 cm to 0.73 cm, that formed due to the antibacterial dressing against Staphylococcus aureus is between 0.32 cm and 0.72 cm, and/or that formed due to the antibacterial dressing against Pseudomonas aeruginosa is between 0.04 cm and 0.52 cm.

FIG. 2 shows the statistical inhibition results of the antibacterial dressing prepared in Example 1 against (A) Escherichia coli, (B) Staphylococcus aureus, and (C) Pseudomonas aeruginosa. FIG. 2 shows the statistical results of the data obtained from FIG. 1. In FIG. 2, the vertical axis indicates the width of the bacteriostatic circle. FIG. 2 shows that the collagen dressing including PHMB, zinc chloride, and magnesium chloride can have inhibitory effects simultaneously against Escherichia coli, Staphylococcus aureus, and Pseudomonas aeruginosa.

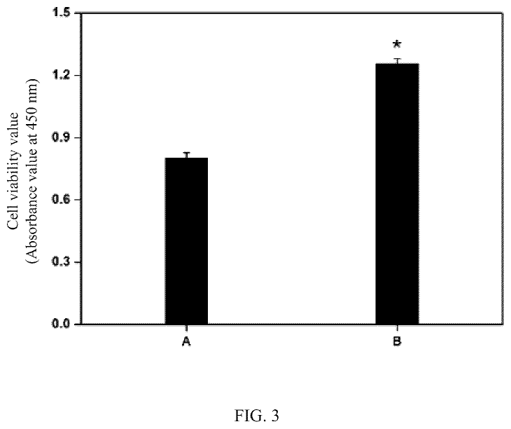

FIG. 3 shows the cytotoxicity results of the antibacterial dressings prepared in Example 1 and the blank control group, in which “A” represents the pure collagen dressing (i.e., the blank control group, including none of PHMB, zinc chloride, and magnesium chloride), “B” represents the antibacterial dressing prepared in Example 1, and “*” indicates that the antibacterial dressing prepared in Example 1 is statistical significant different from the pure collagen dressing.

### 3. Cell Morphology

The collagen antibacterial dressing samples prepared in Examples 1 to 12 and Comparative Examples 1 to 4 are respectively placed in 24-well culture plates, inoculated with a single cell suspension of fibroblasts, cultured in an 37° C. incubator filling with 5% carbon dioxide for 72 hours, then fixed with paraformaldehyde, successively dehydrated with gradient ethanol (50%, 75%, 90%, and 100%), vacuum-dried, sputter-coated with gold, and finally observed under a scanning electron microscope.

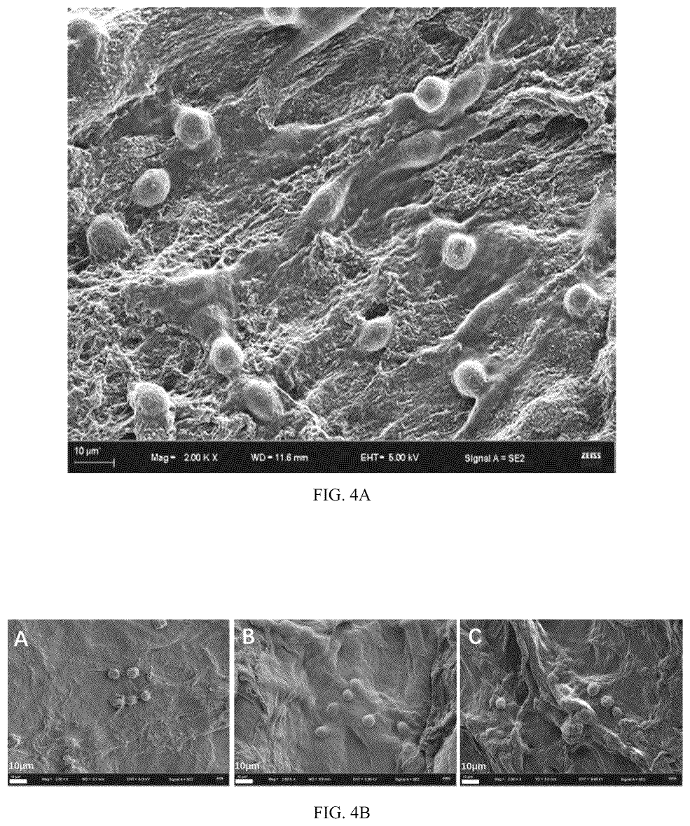

FIG. 4A shows the morphology of the fibroblasts cultured on the antibacterial dressing prepared in Example 1. It can be seen from the morphology that the fibroblasts are of long spindle shape and oriented, forming a layer of extracellular matrix. This suggests a favorable environment for recruiting, migrating, and growing of the fibroblasts, ultimately facilitating the rapid healing of wounds. The morphology of the fibroblasts cultured on the antibacterial dressings prepared in other examples is similar to that cultured on the antibacterial dressing prepared in Example 1, which will not be shown repeatedly.

FIG. 4B shows the morphologies of the fibroblasts cultured on the antibacterial dressings prepared in the comparative examples, in which “A” represents the morphology of the fibroblasts cultured on the antibacterial dressing prepared in Comparative Example 1, “B” represents the morphology of the fibroblasts cultured on the antibacterial dressings prepared in Comparative Examples 2 and 3, and “C” represents the morphology of the fibroblasts cultured on the antibacterial dressings prepared in Comparative Examples 8 and 11 (excessive zinc chloride) and 10 (excessive magnesium chloride).

The results reveal that the fibroblasts have different morphologies on different collagen antibacterial dressings. When cultured on the surface of the collagen antibacterial dressing whose antibacterial layer includes only PHMB, as prepared in Comparative Example 1, the fibroblasts are of spherical shape. This observation validates the cytotoxicity of PHMB. When zinc chloride or magnesium chloride is introduced into the antibacterial layer of the collagen antibacterial dressing upon PHMB (Comparative Examples 2 and 3), the fibroblasts spread out and form an extracellular matrix layer. When the antibacterial layer includes PHMB, zinc chloride, and magnesium chloride in a certain ratio (Example 1), the fibroblasts are of long spindle shape and oriented, forming the extracellular matrix layer. This suggests that when the antibacterial layer includes PHMB, zinc chloride, and magnesium chloride, the antibacterial dressing is more favorable for cell recruitment and migration on the wound surface. Properly increasing the content of zinc chloride and/or magnesium chloride can promote cell spreading and migration, but excessive addition of zinc chloride and/or magnesium chloride can cause significant shrinkage of fibroblasts.

The technical features of the embodiments as described above can be combined arbitrarily. In order to make the descriptions concise, not all possible combinations of the technical features are described in the embodiments. However, as long as there is no contradiction in the combination of these technical features, the combinations shall be considered as in the scope of the present disclosure.

The embodiments as described above are only several implementations of the present disclosure in order to facilitate specific and detailed understanding of the technical solutions of the present disclosure, but they shall not be construed as limiting the scope of the present disclosure. It shall be noted that various modifications and improvements can be made without departing from the concept of the present disclosure for those skilled in the art, and all fall within the protection scope of the present disclosure. It shall be understood that the technical solutions obtained by those skilled in the art via logical analysis, reasoning or limited experimentation based on the technical solutions provided in the present disclosure are all within the protection scope of the appended claims of the present disclosure. Therefore, the protection scope of the patent right of the present disclosure shall be determined by the terms of the claims, and the descriptions and the drawings may be used to interpret the content of the claims.

What is claimed is:

1. An antibacterial dressing, comprising a dressing substrate and an antibacterial layer applied to the dressing substrate, wherein a raw material of the antibacterial layer comprises an antibacterial composition, the antibacterial composition comprises a biguanidine antibacterial agent, a zinc-containing compound, and a magnesium-containing compound with a mass ratio of (0.02 to 1):(0.1 to 0.5):(1 to 5).

2. The antibacterial dressing according to claim 1, wherein the mass ratio of the biguanidine antibacterial agent, the zinc-containing compound, and the magnesium-containing compound is (0.02 to 0.5):(0.2 to 0.5):(2 to 5).

3. The antibacterial dressing according to claim 1, wherein the biguanidine antibacterial agent is selected from the group consisting of polyhexamethylene biguanidine hydrochloride, chlorhexidine, dodine, and any combination thereof.

4. The antibacterial dressing according to claim 1, wherein the zinc-containing compound is selected from the group consisting of zinc chloride, zinc sulfate, zinc acetate, zinc nitrate, and any combination thereof.

5. The antibacterial dressing according to claim 1, wherein the magnesium-containing compound is selected from the group consisting of magnesium chloride, magnesium sulfate, magnesium phosphate, magnesium nitrate, and any combination thereof.

6. The antibacterial dressing according to claim 1, wherein a material of the dressing substrate comprises collagen.

7. The antibacterial dressing according to claim 1, wherein a water-absorption ratio of the dressing substrate is less than or equal to 52.71.

8. The antibacterial dressing according to claim 1, wherein an absorbance value of supernatant at 450 nm in a cell viability test of the antibacterial dressing is above 0.857.

9. The antibacterial dressing according to claim 1, wherein the absorbance value is between 0.86 and 1.37.

10. The antibacterial dressing according to claim 1, being configured to form a bacteriostatic circle surrounding the antibacterial dressing when placing the antibacterial dressing into a culture medium inoculated with Escherichia coli, Staphylococcus aureus, or Pseudomonas aeruginosa, and the bacteriostatic circle is semi-transparent.

11. The antibacterial dressing according to claim 10, wherein after incubated at 37° C. for 24 hours, a width of the bacteriostatic circle formed due to the antibacterial dressing against Escherichia coli is between 0.11 cm to 0.73 cm, that formed due to the antibacterial dressing against Staphylococcus aureus is between 0.32 cm and 0.72 cm, and/or that formed due to the antibacterial dressing against Pseudomonas aeruginosa is between 0.04 cm and 0.52 cm.

12. The antibacterial dressing according to claim 1, being capable of allowing fibroblasts of long spindle shape and oriented to be cultured thereon, forming a layer of extracellular matrix.

13. The antibacterial dressing according to claim 1, wherein the antibacterial dressing is a part of a material or a medicament for promoting wound healing.

14. A method for preparing the antibacterial dressing according to claim 1, comprising the following steps:
dissolving the antibacterial composition to obtain a composite solution comprising free zinc ions, free magnesium ions and a biguanidine antibacterial agent; and
applying the composite solution to a dressing substrate and drying to obtain the antibacterial dressing.

15. The method for preparing the antibacterial dressing according to claim 14, wherein a pH value of the composite solution is ranged from 4.5 to 6.5.

16. The method for preparing the antibacterial dressing according to claim 14, wherein the mass of the biguanidine antibacterial agent is 0.02% to 1% of the mass of the composite solution, the mass of the zinc-containing compound is 0.1% to 0.5% of the mass of the composite solution, and the mass of the magnesium-containing compound is 1% to 5% of the mass of the composite solution.

17. The method for preparing the antibacterial dressing according to claim 14, wherein the step of applying the composite solution to the dressing substrate is comprises a step of immersing the dressing substrate in the composite solution for 30 min to 360 min.

18. The method for preparing the antibacterial dressing according to claim 17, wherein a mass ratio of the dressing substrate to the composite solution is 1:17 to 1:52.71.

19. The method for preparing the antibacterial dressing according to claim 14, wherein a type of drying is selected from the group consisting of natural drying, blast drying, freeze drying, vacuum drying, critical point drying, and any combination thereof.

20. A method for using of the antibacterial dressing according to claim 1, comprising applying the antibacterial dressing onto a skin wound for promoting wound healing.
